(19) United States
(12) Patent Application Publication
(10) Pub. No.: US 2012/0259419 A1
(43) Pub. Date: Oct. 11, 2012

(54) METHOD AND APPARATUS FOR THE TREATMENT OF METATARSOPHALANGEAL JOINT DEGENERATIVE ARTHRITIS

(76) Inventors: Michael Glyn Brown, Houston, TX (US); Jeffrey F. Seavey, Houston, TX (US); Stephen L. Barrett, Phoenix, AZ (US)

(21) Appl. No.: 13/079,901

(22) Filed: Apr. 5, 2011

(51) Int. Cl. A61F 2/42

(52) U.S. Cl. 623/21.19

(57) ABSTRACT

An implantable device includes a prosthetic, an anchor having an outer surface forming a threaded structure, and a retainer securing the anchor to the prosthetic, the retainer limiting the relative movement between the anchor and the prosthetic to rotation of the anchor relative to the prosthetic. A method of repairing the first metatarsophalangealjoint includes cutting away a portion of the tissue of the proximal phalanx to form a cavity, driving an anchor of the implantable device into the cavity to anchor the implantable device to the proximal phalanx, and while the anchor is being driven into the cavity, aligning a prosthetic of the implantable device to the phalanx such that the alignment of the prosthetic is maintained.

### BACKGROUND

The present disclosure relates generally to an implant device and method for implanting the device. More specifically, the present disclosure relates to a hemiarthroplasty implant device for the metatarsophalangeal joint and a method for implanting the same.

The first metatarsophalangeal joint (MPJ) of the foot is susceptible to various diseases which affect the proper functioning of the MPJ. Diseases that cause deterioration of the functioning of the MPJ include osteoarthritis and rheumatoid arthritis. The arthritis may cause the MPJ to stiffen (hallux rigidus) or to become deformed (hallux valgus).

Hallux rigidus is characterized by pain and reduced motion at the first MPJ. Hallux valgus is characterized by deviation of the big toe toward the midline of the foot. Exercise, physiotherapy, and orthotic devices are all used to treat diseases and conditions of the hallux. Additional treatments include non-steroidal drug therapy and steroid injections to alleviate the discomfort of the conditions.

MPJ arthrodesis (fusion) is a standard surgical technique for the treatment of degenerative arthritis of the MPJ as this permits relief of the painful condition, but negatively affects the functioning of the foot. Cheiloectomy (removal of bone irregularities) of the joint is another approach to alleviating the issues associated with the MPJ of the hallux. Excision of the joint by use of the Keller procedure is still another intervention that provides relief to the degenerated MPJ.

The use of prosthetic inter-positional arthroplasty provides relief and may provide an individual with a degenerative MPJ with restored motion in the joint. It is desirable, during arthroplasty, to achieve alignment of the prosthetic device to maintain the proper relationship between the phalange and the metatarsal so that the proper range of motion can be achieved, and the toe is in an acceptable position.

### SUMMARY

The present application discloses one or more of the features recited in the appended claims and/or the following features which, alone or in any combination, may comprise patentable subject matter:

According to a first aspect of the present disclosure, an implantable device for the first metatarsophalangeal joint may include an articulating surface prosthetic, an anchor, and a retainer. The anchor may include a longitudinal axis and an outer surface that forms a threaded structure. The anchor may also include a cavity aligned on the longitudinal axis and configured to receive a driver to engage the anchor to rotate the anchor about the longitudinal axis. The retainer may be engaged with the articulating surface prosthetic and the anchor to prevent movement of the articulating surface prosthetic relative to the anchor along the longitudinal axis of the anchor. The retainer may permit rotation of the anchor about the longitudinal axis of the anchor relative to the articulating surface prosthetic.

The articulating surface prosthetic may include a barb to secure the prosthetic to tissue when the implantable device is installed.

The articulating surface prosthetic may include a first surface and a second surface. The barb may extend from the first surface. The second surface may form a recess shaped to engage a surface of the metatarsal base.

The anchor may include an annular groove circumscribing the body of the anchor. The retainer may engage the annular groove. The groove may form a semi-circular channel. The retainer may be a pin circular cross-section. The pin may be secured to the prosthetic and sized to allow the anchor to move relative to the prosthetic with no movement of the prosthetic along the longitudinal axis of the anchor.

The anchor may include a cannulated body having a passageway through the cannulated body. The passageway may be sized to be positioned on a locator secured to the tissue. The driver receiving cavity may be accessible while the cannulated body is positioned on the locator.

The articulating surface prosthetic may permanently replace a portion of the proximal phalanx after the implantable device is installed.

In another aspect of the disclosure, an apparatus for repairing the first metatarsophalangeal joint may include an implantable device for replacing at least a portion of a bone structure at the first metatarsophalangeal joint, a cutter for preparing the bone structure for installation of the implantable device, and a driver for installing the implantable device in the bone structure.

The implantable device may include a prosthetic, an anchor having an outer surface forming a threaded structure and configured to receive the driver to engage the anchor to rotate the anchor, and a retainer securing the anchor to the prosthetic, the retainer limiting the relative movement between the anchor and the prosthetic to rotation of the anchor relative to the prosthetic.

The prosthetic may include a concave surface defining a recess shaped to engage a surface of the metatarsal base when the implantable device is installed.

The anchor may include a cannulated body having a passageway through the cannulated body. The passageway may be sized to be positioned on a locator secured to the bone structure. The driver receiving cavity may be accessible while the cannulated body is positioned on the locator.

According to still another aspect of the disclosure a method of repairing the first metatarsophalangeal joint may include resecting a portion of the proximal phalanx, positioning a drill guide on the base of the proximal phalanx, and driving a locator through the drill guide into tissue of the proximal phalanx. The method may also include positioning a cutter on the locator, cutting away a portion of the tissue of the phalanx to form a cavity, and positioning an implantable device on the locator. The method still further may include driving an anchor of the implantable device into the cavity to anchor the implantable device to the phalanx, and while the anchor is being driven into the cavity, aligning a prosthetic of the implantable device to the phalanx such that as the alignment of the prosthetic is maintained.

The step of aligning the prosthetic of the implantable device may include rotating the anchor relative to the prosthetic device.

The step of aligning the prosthetic of the implantable device may include limiting movement of the prosthetic in a direction parallel to the longitudinal axis of the anchor.

The method may further include removing the locator.

The step of cutting away a portion of the tissue of the phalanx to form a cavity may include broaching the tissue.

The step of aligning a prosthetic of the implantable device to the phalanx may include driving a barb of the prosthetic device into tissue of the proximal phalanx.

Additional features, which alone or in combination with any other feature(s), including those listed above and those listed in the claims, may comprise patentable subject matter and will become apparent to those skilled in the art upon consideration of the following detailed description of illustrative embodiments exemplifying the best mode of carrying out the invention as presently perceived.

### BRIEF DESCRIPTION OF THE DRAWINGS

The detailed description particularly refers to the accompanying figures in which:

FIG. 1 is a perspective view of an implantable device;

FIG. 2 is an exploded perspective view of the implantable device of FIG. 1;

FIG. 3 is a plan view of an anchor of the implantable device of FIG. 1;

FIG. 4 is a cross-sectional view of the anchor of FIG. 3 taken along line 4-4 in FIG. 3;

FIG. 5 is a top view of the implantable device of FIG. 1;

FIG. 6 is a diagrammatic view of the bone structure of a human foot with the implantable device implanted;

FIG. 7 is a perspective view of an instrument used to install the implantable device of FIG. 1;

FIG. 8 is a perspective view of the instrument of FIG. 7 positioned on the base of a proximal phalanx of a human;

FIG. 9 is a perspective view of the instrument of FIGS. 7 and 8 with a locator driven into the proximal phalanx using the instrument of FIG. 7 to guide the positioning of the locator;

FIG. 10 is a perspective view of another instrument used to prepare the proximal phalanx for installation of the implantable device of FIG. 1;

FIG. 11 is an enlarged view of a portion of the instrument of FIG. 10; and

FIG. 12 is a perspective view of yet another instrument used to install the implantable device of FIG. 1.

### DETAILED DESCRIPTION OF THE DRAWINGS

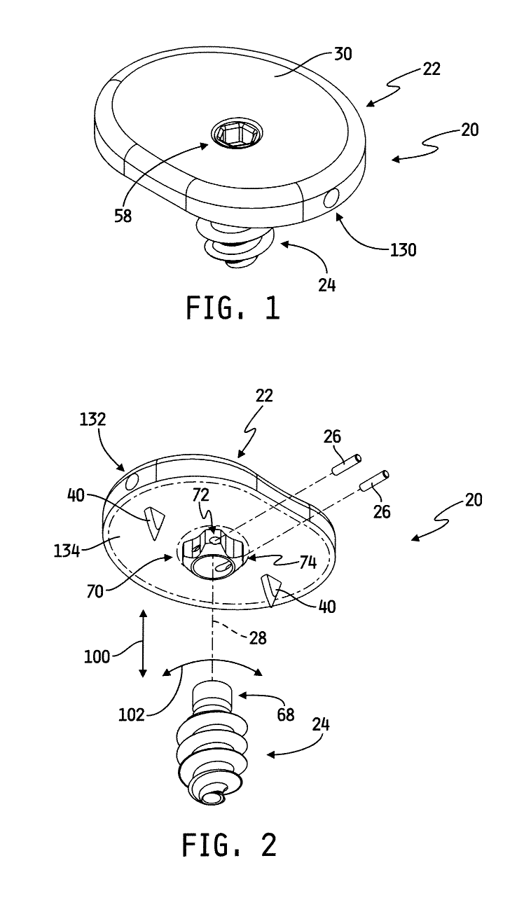

An implant 20 suitable for treatment and correction of diseases of the metatarsophalangeal joint (MPJ) of the hallux (big toe) includes an articulating surface prosthetic 22 and an anchor screw 24 as shown in FIGS. 1 and 2. The anchor screw 24 is secured to the articulating surface prosthetic 22 by a pair of pins 26. The anchor screw 24 is free to rotate relative to the articulating surface prosthetic 22 about an axis 28 when the anchor screw 24 is engaged with the articulating surface prosthetic 22. The articulating surface prosthetic 22 is restrained from movement along the axis 28 in the direction of arrow 100 by the pins 26 such that when the implant 20 is assembled, the only relative movement between the anchor screw 24 and articulating surface prosthetic 22 is rotational movement about the axis 28 as indicated by arrow 102. As will be discussed in further detail below, the freedom of relative movement about axis 28 allows the articulating surface prosthetic 22 to be aligned with a patient's anatomy during installation while also permitting full engagement of the anchor screw 24 with the bone into which the anchor screw 24 is being inserted.

In the illustrative embodiment, the implant 20 is used as a prosthetic for the phalanx in the MPJ. The articulating surface prosthetic 22 includes a concave surface 30 which is shaped to engage the head 34 of the first metatarsal 32 shown in FIG. 6. The implant 20 shown in phantom in FIG. 6 is secured to the proximal phalanx 36 of the hallux 38 by engaging the anchor screw 24 with the proximal phalanx 36. Proper alignment of the concave surface 30 with the head 34 of the first metatarsal 32 is necessary for the proper repair of the MPJ. In FIG. 6, a medial view of the hallux 38 of the left foot of an individual shows that the proximal phalanx 36 moves about the head 34 of the first metatarsal 32 as indicated by arrow 104.

Referring again now to FIG. 1, the concave surface 30 of the articulating surface prosthetic 22 is lobe-shaped so that proper alignment with the proximal phalanx 36 to provide the maximum range of motion after the repair. During installation of the implant 20, a pair of barbs 40 (seen in FIG. 2) which extend from the underside of the articulating surface prosthetic 22, are driven into the proximal phalanx 36 to prevent rotation of the articulating surface prosthetic 22 relative to proximal phalanx 36. The underside of articulating prosthetic surface 22 may include an area 134 coated with a surface treatment to promote bony in-growth. In one embodiment, the area 134 is treated with titanium plasma. The relative movement of the anchor screw 24 relative to the articulating surface prosthetic 22 during the installation improves the opportunity for proper installation and reduces the time of installation.

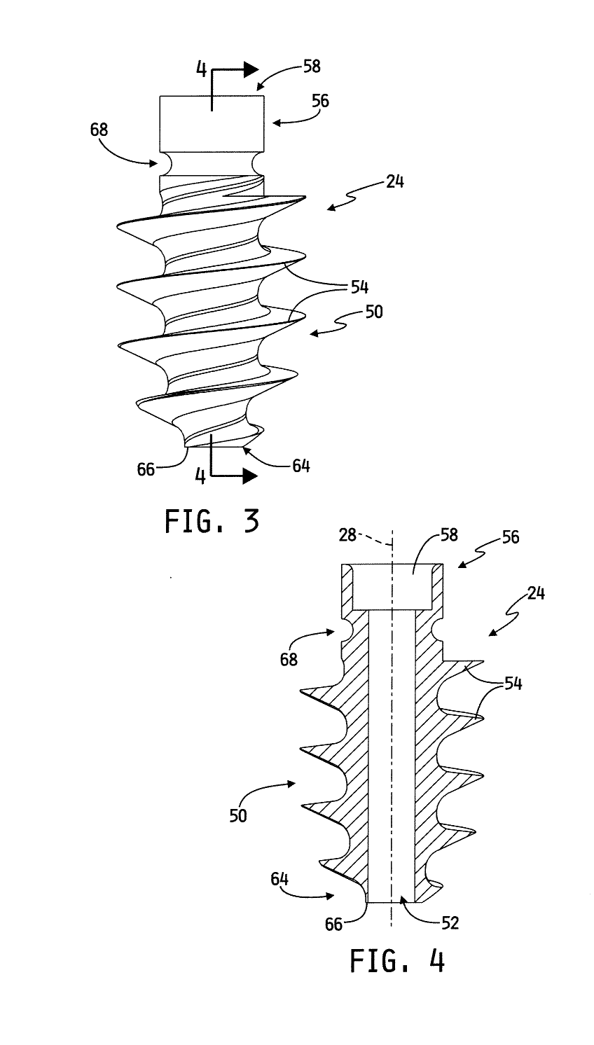

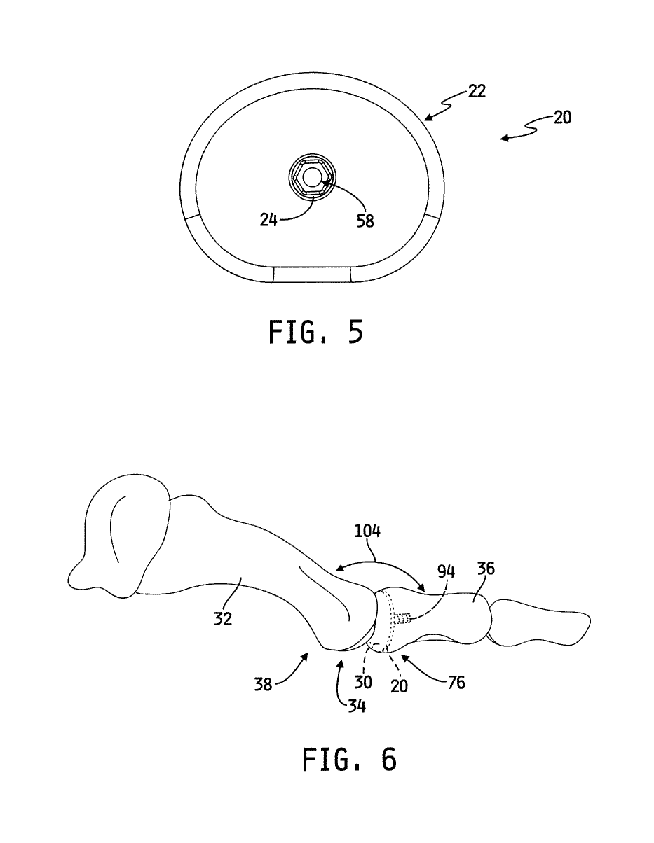

Referring now to FIGS. 3 and 4, the anchor screw 24 is formed to include a cannulated body 50 having a passage 52 through the cannulated body 50 and threads 54 formed on the exterior of the cannulated body 50. An end 56 of the anchor screw 24 includes a hex socket 58 formed therein and configured to receive a head 62 of a hexagonal driving tool 60 shown in FIG. 12. The end 64 opposite end 56 of the anchor screw 24 has a generally flat surface 66. The anchor screw 24 includes an annular groove or channel 68 formed about the exterior of the cannulated body 50 and positioned just below the hex socket 58. Referring to FIG. 2, the anchor screw 24 is inserted into a stem 70 formed on the bottom of the articulating surface prosthetic 22. The stem 70 has two passages 72, 74 formed on either side of the stem 70 and sized to receive the pins 26. When the anchor screw 24 is inserted into the stem 70, the pins 26 pass through the respective passages 72, 74 and a portion of the pins 26 are received in or engage the annular channel 68. The pins 26 then retain the anchor screw 24 to the articulating surface prosthetic 22 to form the implant 20. The clearance between the anchor screw 24 and the stem 70 and the clearance between the pins 26 and the annular channel 68 are sufficient to permit the anchor screw 24 to rotate freely about the axis 28 relative to the articulating surface prosthetic 22. This relative movement allows the articulating surface prosthetic 22 to be held in the proper orientation while the anchor screw 24 is driven into the proximal phalanx 36.

The process of repairing the first MPJ with the implant 20 illuminates the benefits of the implant 20. Upon exposure of the first MPJ, the surgeon exposes the proximal phalangeal base 76. The surgeon then addresses degenerative changes of the head 34 including drilling of deficits and surgical correction of metatarsal elevatus or long first metatarsal, for example. Once the first metatarsal 32 is repaired, the proximal phalangeal base 76 is resected by about 3 mm, for example. During the osteotomy of the proximal phalangeal base 76, the proximal phalangeal base 76 is angularly corrected.

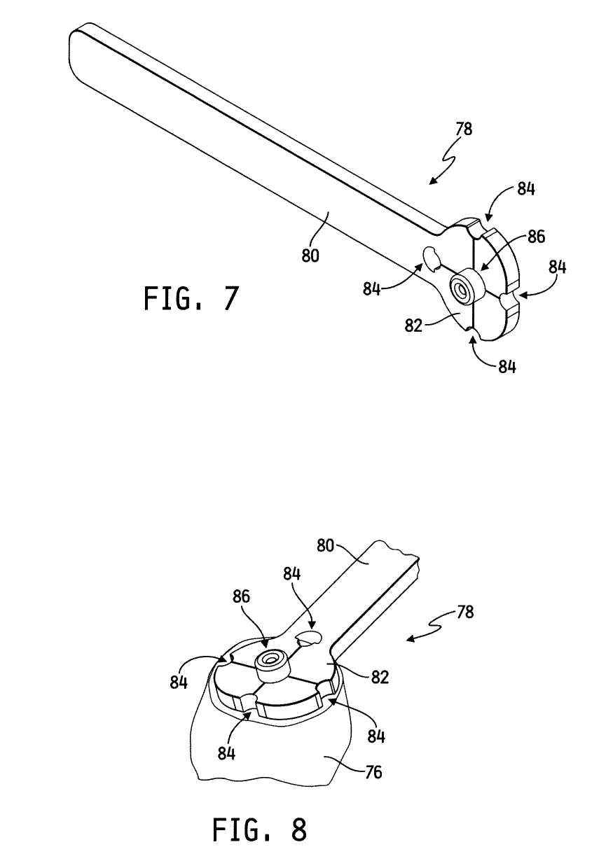

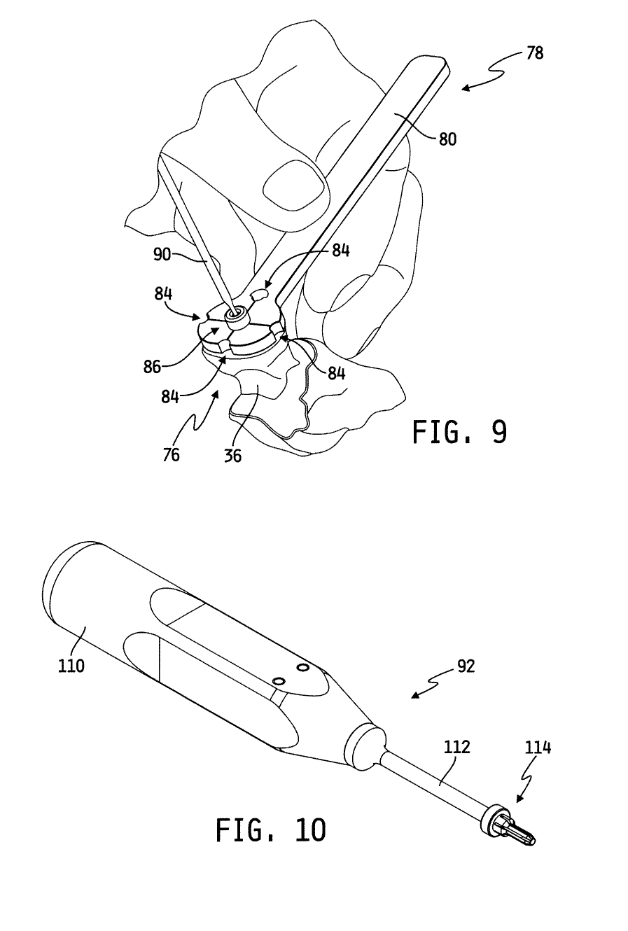

Upon completion of the resection and correction, the proximal phalangeal base 76 is sized to determine the appropriate size for implant 20 to be used. Referring now to FIGS. 7 and 8, a sizer 78 is placed on the resected proximal phalangeal base 76. It should be understood that during a procedure, a number of different sizers 78 will be available. The surgeon places different sizes of sizer 78 on the proximal phalangeal base 76 until the appropriate sizer 78 is selected. The sizer 78 includes a handle 80 and a template 82. The profile of the template 82 is the same as a like-sized articulating surface prosthetic 22. Four notches 84 positioned about the perimeter of the template 82 allow a surgeon to align the sizer 78 to determine that the correct size is chosen. Similarly, the sizer 78 acts to locate the implant 20.

Proper location for the implant 20 is defined by a drill guide 86 positioned on the template 82 of the sizer 78. With the sizer 78 properly positioned, a surgeon drives a 1.5 mm k-wire 90 into the proximal phalanx 36 using the drill guide 86 to guide the k-wire 90 as it is driven. Illustratively, the k-wire 90 is driven approximately 40 mm or to the level of the interphalangeal joint. The k-wire 90 is driven using a standard driver as is known in the art.

Once the k-wire 90 is inserted, the sizer 78 is removed with the k-wire 90 in place. A hand broach 92 shown in FIG. 10 is positioned over the k-wire 90 and with the k-wire 90 as a guide, the proximal phalangeal base 76 is manually broached to form a cavity 94 (seen in FIG. 6) in which the anchor screw 24 is inserted during installation of the implant 20.

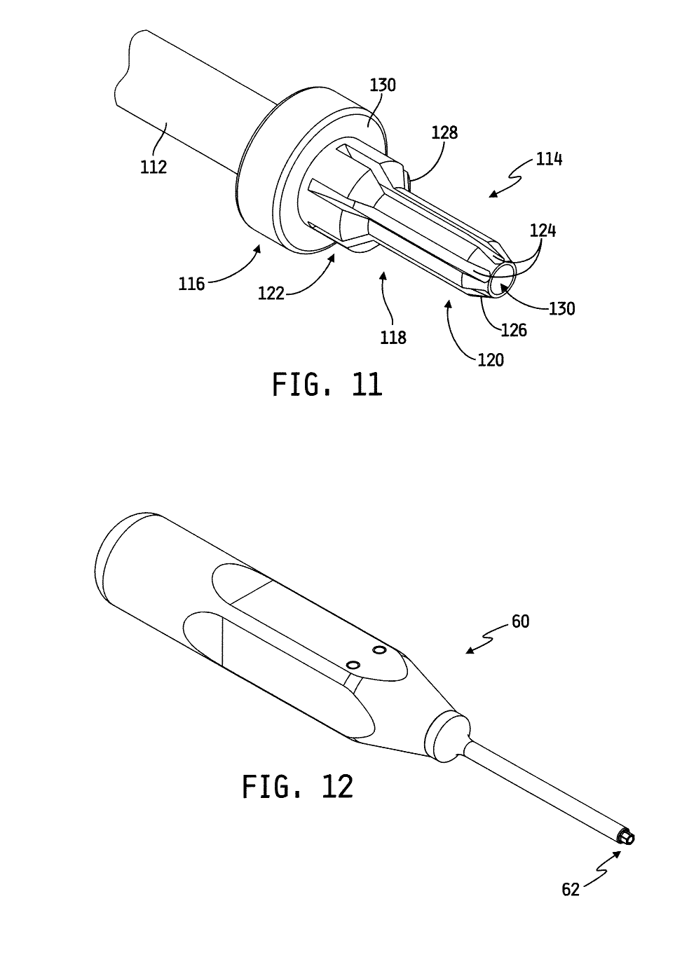

The hand broach 92 includes a handle 110, a stem 112, and a cutting head 114. The cutting head 114, seen in detail in FIG. 11, includes a stop 116 and a cutter 118 that extends from the stop 116. The cutter 118 includes a small diameter portion 120 and a large diameter portion 122. The hand broach 92 is cannulated with a passage 30 that passes through the length of the hand broach 92. A leading edge 126 of the cutter 118 is used to open the cavity 94. Rotating the broach 92 causes flutes 124 formed on the cutter 118 to cut away bone tissue of the proximal phalanx 36. Once the smaller diameter portion 120 is worked into the bone tissue, a leading edge 128 works to widen the cavity such that the larger diameter portion 122 clears space for the stem 70 in the proximal phalanx 36.

Once the cavity 94 is formed, the implant 20 is positioned over the k-wire 90 with the k-wire 90 passing through the cannulation 52 of the anchor screw 24. Once the anchor screw 24 is in position at the surface of the proximal phalangeal base 76, the articulating surface prosthetic 22 is aligned with the proximal phalanx 36. A cannulated driver 96 shown in FIG. 12 is positioned over the k-wire 90 and the hexagonal head 98 of the driver 96 is positioned in the hex socket 58. The anchor screw 24 is then manually driven into the proximal phalanx 36. As the anchor screw 24 is driven, the prosthetic is manually positioned in the proper alignment with the long axis of the proximal phalanx 36. Upon installation of the implant 20, the driver 96 and k-wire 90 are removed.

Because the anchor screw 24 rotates relative to the articulating surface prosthetic 22, the surgeon may position the articulating surface prosthetic 22 and securely anchor the implant in the tissue of the proximal phalanx 36 without having to time the rotations of the anchor screw 24 to align the articulating surface prosthetic 22. In addition, the independent movement of the anchor screw 24 permits the barbs 40 to be driven into the proximal phalangeal base 76 and seat without undue tissue disruption. The articulating surface prosthetic 22 includes two recesses 130 and 132 formed in an outer edge. The surgeon may position a piece of k-wire or other instrument into one of the recesses 130 or 132 using the k-wire to maintain the proper orientation of the articulating surface prosthetic 22 during the driving of the anchor screw 24.

In the illustrative embodiment, the width of the articulating surface prosthetic 22 is about 19 mm and the height of the articulating surface prosthetic 22 is about 15 mm. It should be understood that a number of different sizes of prosthetic articulating surface prosthetic 22 may be available and used for various sizes of patients. The illustrative implant 20, hand broach 92, and driver 96 are all available as part of the Spiral Lok™ family of products from Instratek, Inc. of Spring, Tex.

While the illustrative embodiment is used for repair of the MPJ, those of ordinary skill in the art will readily recognize the applicability of this disclosure to other implants including a prosthetic anchored to bone tissue. The relative movement of the anchor to the prosthetic while the implant is installed allows for improved alignment of the prosthetic with skeletal landmarks or features.

Although certain illustrative embodiments have been described in detail above, variations and modifications exist within the scope and spirit of this disclosure as described and as defined in the following claims.

1. An implantable device for the first metatarsophalangeal joint comprising
an articulating surface prosthetic,
an anchor including a longitudinal axis, the anchor having an outer surface forming a threaded structure and a cavity aligned on the longitudinal axis, the cavity being configured to receive a driver to engage the anchor to rotate the anchor about the longitudinal axis,
a retainer engaged with the articulating surface prosthetic and the anchor to prevent movement of the articulating surface prosthetic relative to the anchor along the longitudinal axis of the anchor and permit rotation of the anchor about the longitudinal axis of the anchor relative to the articulating surface prosthetic.

2. The implantable device of claim 1, wherein the articulating surface prosthetic includes a barb to secure the prosthetic to tissue when the implantable device is installed.

3. The implantable device of claim 2, wherein the articulating surface prosthetic includes a first surface, the barb extending from the first surface, and a second surface forming a recess shaped to engage a surface of the metatarsal base.

4. The implantable device of claim 3, wherein the anchor includes an annular groove circumscribing the body of the anchor and the retainer engages the annular groove.

5. The implantable device of claim 4, wherein the groove forms a semi-circular channel and the retainer is a pin having circular cross-section, the pin secured to the prosthetic and sized to allow the anchor to move relative to the prosthetic with minimal movement of the prosthetic along the longitudinal axis of the anchor.

6. The implantable device of claim 1, wherein the anchor includes an annular groove circumscribing the body of the anchor and the retainer engages the annular groove.

7. The implantable device of claim 6, wherein the groove forms a semi-circular channel and the retainer is a pin with a circular-cross-section, the pin secured to the prosthetic and sized to allow the anchor to move relative to the prosthetic with minimal movement of the prosthetic along the longitudinal axis of the anchor.

8. The implantable device of claim 7, wherein the anchor includes a cannulated body having a passageway through the cannulated body, the passageway sized to be positioned on a locator secured to the tissue, and wherein the driver receiving cavity is accessible while the cannulated body is positioned on the locator.

9. The implantable device of claim 8, wherein the articulating surface prosthetic includes a concave surface defining a recess shaped to engage a surface of the metatarsal base when the implantable device is installed.

10. The implantable device of claim 9, wherein the articulating surface prosthetic permanently replaces a portion of the proximal phalanx after the implantable device is installed.

11. An apparatus for repairing the first metatarsophalangeal joint comprising
an implantable device for replacing at least a portion of a bone structure at the first metatarsophalangeal joint,
a cutter for preparing the bone structure for installation of the implantable device, and
a driver for installing the implantable device in the bone structure.

12. The apparatus of claim 11, wherein the implantable device comprises
a prosthetic,
an anchor having an outer surface forming a threaded structure and configured to receive the driver to engage the anchor to rotate the anchor, and
a retainer securing the anchor to the prosthetic, the retainer limiting the relative movement between the anchor and the prosthetic to rotation of the anchor relative to the prosthetic.

13. The apparatus of claim 12, wherein the prosthetic includes a concave surface defining a recess shaped to engage a surface of the metatarsal base when the implantable device is installed.

14. The apparatus of claim 13, wherein the anchor includes a cannulated body having a passageway through the cannulated body, the passageway sized to be positioned on a locator secured to the bone structure, and wherein the driver receiving cavity is accessible while the cannulated body is positioned on the locator.

15. A method of repairing the first metatarsophalangealjoint comprising the steps of:
resecting a portion of the proximal phalanx;
positioning a drill guide on the base of the proximal phalanx;
driving a locator through the drill guide into tissue of the proximal phalanx;
positioning a cutter on the locator;
cutting away a portion of the tissue of the phalanx to form a cavity;
positioning an implantable device on the locator;
driving an anchor of the implantable device into the cavity to anchor the implantable device to the phalanx; and
while the anchor is being driven into the cavity, aligning a prosthetic of the implantable device to the phalanx such that the alignment of the prosthetic is maintained.

16. The method of claim 15, wherein the step of aligning the prosthetic of the implantable device includes rotating the anchor relative to the prosthetic device.

17. The method of claim 16, wherein the step of aligning the prosthetic of the implantable device includes limiting movement of the prosthetic in a direction parallel to the longitudinal axis of the anchor.

18. The method of claim 17, further comprising the step of removing the locator.

19. The method of claim 18, wherein the step of cutting away a portion of the tissue of the phalanx to form a cavity includes broaching the tissue.

20. The method of claim 19, wherein the step of aligning a prosthetic of the implantable device to the phalanx includes driving a barb of the prosthetic device into tissue of the proximal phalanx.
